(19) United States
(12) Patent Application Publication
(10) Pub. No.: US 2007/0077268 A1
(43) Pub. Date: Apr. 5, 2007

(54) Hydrophobic carrier modified implants for beneficial agent delivery

(75) Inventors: Richard S. King, Warsaw, IN (US); Mark D. Hanes, Winona Lake, IN (US); Sarah Aust, Warsaw, IN (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: DePuy Products, Inc., Warsaw, IN (US)

(21) Appl. No.: 11/241,136

(22) Filed: Sep. 30, 2005

(51) Int. Cl. A61F 2/00; A61K 31/015

(52) U.S. Cl. 424/423; 514/763

(57) ABSTRACT

Disclosed is a bearing material for a medical implant or medical implant part, the bearing material comprising: (a) a matrix of crosslinked polyethylene, (b) a biocompatible hydrophobic carrier, and (c) a beneficial agent soluble in the biocompatible hydrophobic carrier. Also disclosed are methods for preparing a bearing material in accordance with the invention. The bearing material releases a beneficial agent at least partially by a load-activated mechanism.

### BACKGROUND OF THE INVENTION

Implants have been used to replace parts of the human body, e.g., the hip, the knee, and the extremity joints. Post-surgery pain and infection are some of the medical concerns in orthopaedic joint replacements. Accordingly, attempts have been made to deliver a beneficial agent such as a drug by the use of coated implants wherein the coating includes a drug. For example, implants coated with a polyurethane coating or polymethyl methacrylate bone cement coating have been used in orthopaedic applications to deliver drugs to the site of pain or infection. These implants, however, have one or more drawbacks. There is a desire to improve upon one or more properties of such implants, particularly the beneficial agent release profile. The present invention provides such an implant.

### BRIEF SUMMARY OF THE INVENTION

In an implant, the bearing material having a bearing surface is paired with an opposing metal or ceramic component. The bearing surface is also called the articulating surface. The invention provides a bearing material for a medical implant or medical implant part, the bearing material comprising: (a) a matrix of crosslinked polyethylene, (b) a biocompatible hydrophobic carrier, and (c) a beneficial agent soluble in the biocompatible hydrophobic carrier. An advantage of the bearing material of the invention is that the beneficial agent release profile is at least partially load activated. The beneficial agent release mechanism, unlike the prior art devices, is not purely passive. The beneficial agent release from the bearing material of the invention can be based on a combination of load-activated and passive mechanisms. Cyclic loading of the bearing material during use pumps out the hydrophobic carrier and beneficial agent and provides a sustained release of the agent without significantly compromising its mechanical properties, for example, wear resistance.

The invention also provides methods for preparing a bearing material in accordance with the invention. The methods rely on soaking polyethylene in a solution of the beneficial agent in a biocompatible hydrophobic carrier. The biocompatible hydrophobic carrier and the beneficial agent are diffused into the polyethylene. The methods of the invention have one or more of the following advantages. Unlike methods involving melt processing which could lead to degradation, especially when processing high melting and/or thermally sensitive beneficial agents or carriers, it is possible by the methods of the present invention to process with significantly reduced degradation of the beneficial agent or the biocompatible carrier. The methods of the invention also provide more homogeneous or uniform distribution, e.g., improved uniformity of distribution of the carrier and beneficial agent, compared to methods which mold a blend of powdered polyethylene, carrier, and beneficial agent. The methods of the invention do not require the use of solvents which are not biocompatible, e.g., solvents such as isopropyl alcohol, cyclohexane, n-hexane, and benzene.

### BRIEF DESCRIPTION OF THE DRAWINGS

FIG. 1 depicts the weight change of UHMWPE pins as a function of the number of test cycles under cycling loading and without pin movement, as illustrated in Example 1. A-B correspond to the control samples (not containing squalene) and C-D correspond to samples containing squalene.

FIG. 2 depicts the wear rate of UHMWPE pins in mg per million cycles under cyclic loading and with pin movement, as illustrated in Example 2. The light bars correspond to the control samples (not containing squalene) and the dark bars correspond to the test samples containing squalene.

### DETAILED DESCRIPTION OF THE INVENTION

The foregoing need has been fulfilled to a great extent by the present invention, which provides a bearing material for a medical implant or medical implant part, the bearing material comprising: (a) a matrix of crosslinked polyethylene, (b) a biocompatible hydrophobic carrier, and (c) a beneficial agent soluble in the biocompatible hydrophobic carrier.

In accordance with the present invention, any suitable polyethylene can be used. The polyethylene for use in the invention generally possesses a weight average molecular weight of about 10⁵ atomic mass units (amu) or more. Typically, the weight average molecular weight of the polyethylene is generally between about 4×10⁵ to about 10⁷ amu. The polyethylene preferably is an ultrahigh molecular weight polyethylene (UHMWPE). UHMWPE has excellent wear resistance for various modes of loading. The normal shear and compressive loading on UHMWPE articulating surface provides a mechanism for delivery of a beneficial agent to the patient. The UHMWPE can be non-crosslinked, or preferably crosslinked.

The term “ultrahigh molecular weight polyethylene” refers to a polyethylene polymer having a weight average molecular weight of about 400,000 amu or more. Preferably, the UHMWPE has a weight average molecular weight of about 1,000,000 (e.g., about 2,000,000 or about 3,000,000) amu or more. Typically, the weight average molecular weight of the UHMWPE is about 10,000,000 amu or less, more preferably about 6,000,000 amu or less. UHMWPE suitable for use in the invention includes, but is not limited to, commercially available UHMWPE's such as GUR 1050 and GUR 1020 powdered UHMWPE (weight average molecular weight of about 2,000,000 to about 6,000,000 amu) from Ticona (Summit, N.J.).

In accordance with the invention, any suitable biocompatible hydrophobic carrier can be used, for example, the biocompatible hydrophobic carrier is selected from the group consisting of squalane, squalene, benzyl benzoate, fatty acids, glycerides, polyisoprenoids, cholesterol, cholesterol esters, and any combination thereof, particularly squalane, squalene, and benzyl benzoate. Preferably, the biocompatible hydrophobic carrier is a lipid, e.g., those selected from the group consisting of squalane, squalene, fatty acids, glycerides, polyisoprenoids, cholesterol, cholesterol esters, and any combination thereof, particularly squalane and squalene. In accordance with the invention, the bearing material includes the biocompatible hydrophobic carrier in any suitable amount, for example, in an amount from about 0.01 wt. % to about 20 wt. %, preferably from about 0.1 wt. % to about 10 wt. %, and more preferably from about 1 wt. % to about 5 wt. % of the bearing material.

A beneficial agent is one that confers one or more benefits, advantages, or help to the implant recipient or the implant itself. Examples of the benefit, advantage, or help include increased length of life time of use of the implant in the recipient, improved tissue compatibility, and the ability to deliver drugs or other materials to patient. In accordance with the present invention, one or more beneficial agents, e.g., drugs and other materials such as chemicals or biologicals can be included in the bearing material.

A drug is any chemical compound that affects the processes of the human mind or body, for example, a compound used on or administered to humans or animals as an aid in the diagnosis, treatment, or prevention of disease or other abnormal condition, for the relief of pain or suffering, or to control or improve any physiologic or pathologic condition. Any suitable beneficial agent can be employed, e.g., those selected from the group consisting of antibiotics, analgesics, anti-bone resorption drugs, bone growth factors, anti-cancer drugs, antioxidants, and any combination thereof. Examples of chemicals include antioxidants. Antioxidants could reduce or prevent the oxidation and degradation of the articulating surface of the bearing material. Examples of biologicals include a substance made from a living organism or its products. Biologicals may be used to prevent, diagnose, treat or relieve of symptoms of a disease. Examples of biologicals include antibodies, interleukins, and immunomodulators.

The beneficial agent has a solubility of at least about 1% by weight in the hydrophobic carrier, and generally of from about 1% to about 100% or more, and preferably from about 10% to about 100% by weight of the hydrophobic carrier. The beneficial agent can be included in the bearing material in any suitable amount, for example, in an amount of from about 0.001 wt. % to about 20 wt. %, preferably from about 0.01 wt. % to about 10 wt. %, and more preferably from about 0.1 wt. % to about 5 wt. % of the bearing material.

The orthopaedic bearing material of the invention can be prepared by any suitable method, e.g., by diffusing a solution of the beneficial agent in a biocompatible hydrophobic carrier into a polyethylene. For example, an irradiated polyethylene can be soaked in a solution of the beneficial agent. In accordance with an embodiment, the invention provides a method for producing a bearing material for a medical implant or medical implant part, the method comprising:

(a) providing a raw material in consolidated form comprising polyethylene, e.g., ultrahigh molecular weight polyethylene having a weight average molecular weight of about 400,000 atomic mass units or more,

(b) irradiating at least a portion of the raw material to crosslink at least a portion of the polyethylene contained therein and form a matrix of crosslinked polyethylene,

(c) providing a solution comprising a biocompatible hydrophobic carrier and a beneficial agent soluble in the biocompatible hydrophobic carrier, and

(d) contacting at least a portion of the matrix from (b) with the solution to swell the polyethylene and diffuse the biocompatible hydrophobic carrier and the beneficial agent into the matrix.

The raw material in consolidated form is a precursor to the bearing material, which can be of any consolidated shape, e.g., a rod, sheet, preform, or a finished part. The raw material in consolidated form can be prepared by any suitable method, for example, by molding, extrusion, or solvent casting. Alternatively, the raw material in consolidated form can be machined or molded from a block or sheet of a polymer, e.g., of a crosslinkable polymer such as UHMWPE.

Irradiation can be carried out by using any suitable radiation, e.g., ionizing radiation. Ionizing radiation is a radiation, in which an individual particle, e.g., electron, positron, alpha particle, or neutron, carries high enough energy, or an electromagnetic radiation having a high enough energy, to ionize an atom or molecule in the irradiated substrate. Examples of electromagnetic radiation include gamma radiation, X-ray, and ultraviolet light, preferably gamma radiation.

The raw material in consolidated form can be exposed to any suitable amount or dose of radiation, such as from about 1 to about 100 Mrad or more, preferably from about 5 to about 25 Mrad, and more preferably from about 5 to about 10 Mrad. The energy of the radiation is selected so that it is effective to crosslink at least a portion of the exposed surface of the raw material in consolidated form of the bearing material.

Optionally, the raw material in consolidated form after irradiation (or matrix), can be treated suitably so that any free radicals generated during irradiation are reduced or eliminated. An example of such a treatment is heat treatment, e.g., remelting or annealing. Remelting involves heating the irradiated polyethylene above its melting temperature, e.g., from about 1° C. to about 160° C. above the melting temperature of the irradiated polyethylene, preferably from about 40° C. to about 80° C. above the melting temperature. Thus, for example, the remelting temperature for irradiated UHMWPE can be from about 136° C. to about 300° C., preferably from about 136° C. to about 250° C., and more preferably from about 136° C. to about 200° C.

Generally, the remelting temperature is inversely proportional to the remelting period. Polyethylene is preferably remelted over a period from about 1 hour to about 2 days, more preferably from about 1 hour to about 1 day, and most preferably from about 2 hours to about 12 hours. Since, depending on the time and temperature applied, annealing can produce less of an effect than remelting on physical properties such as crystallinity, yield strength and ultimate strength, annealing may be used in place of remelting as a means for reducing or eliminating the free radicals remaining in the polymer after irradiation crosslinking, in order to maintain the physical properties within limits required by the user. Thermal treatment, such as remelting or annealing, removes free radicals and thereby improves long term wear resistance of the polymer.

Annealing involves heating the crosslinked polyethylene below its melting temperature. The annealing temperature can be from about room temperature to below the melting temperature of the irradiated polyethylene; preferably from about 90° C. to about 1° C. below the melting temperature of the irradiated polymer; and more preferably from about 60° C. to about 1° C. below the melting temperature of the irradiated polyethylene. For example, irradiated UHMWPE may be annealed at a temperature from about 25° C. to about 135° C., preferably from about 50° C. to about 135° C., and more preferably from about 80° C. to about 135° C. The annealing period can be from about 2 hours to about 7 days, preferably from about 7 hours to about 5 days, and more preferably from about 10 hours to about 2 days.

The solution comprising the beneficial agent and hydrophobic carrier can optionally include other suitable additives, for example, surfactants, solubilizing agents such as co-solvents or complexing agents, viscosity modifiers, swelling agents, and stabilizing agents.

The irradiated matrix, or portion thereof, e.g., 10, 20, 30, 40, 50, 60, 70, 80, 90, or 100 percent of the matrix, can be contacted with the solution in any suitable method, for example, the matrix is soaked or immersed in the solution. The solution can be maintained at any suitable temperature, for example, at a temperature of about 30° C. to about 150° C., preferably at a temperature of about 50° C. to about 120° C., and more preferably at a temperature of about 60° C. to about 110° C.

The irradiated matrix can be contacted with the solution for any suitable period of time, for example, for a period of from about 0.1 hour or more, such as for about 2 hours or more, preferably from about 8 to about 24 hours, in (d). The contact time can vary with the temperature, for example, inversely with temperature. The amount of solution absorbed will depend on the temperature, contact time, sample size, and surface area.

The matrix produced in (d) can be dried to remove excess biocompatible hydrophobic carrier or therapeutic agent and yield a bearing material having a desired final concentration of biocompatible hydrophobic carrier and beneficial agent.

Optionally, the raw material in consolidated form, e.g., rod, sheet, or preform, can be shaped by suitable method, e.g., machining, a shape and size desired for the bearing material. This can be performed before or after irradiation. The orthopaedic bearing material can be sterilized by a suitable method, e.g., by a non-irradiative method such as the use of ethylene oxide gas.

In another embodiment, irradiation can be carried out after diffusing the beneficial agent and carrier into the raw material in consolidated form. Accordingly, present invention provides a method for producing a bearing material for a medical implant or medical implant part, the method comprising:

(a) providing a raw material in consolidated form comprising polyethylene, e.g., ultrahigh molecular weight polyethylene having a weight average molecular weight of about 400,000 atomic mass units or more,

(b) providing a solution comprising a biocompatible hydrophobic carrier and a beneficial agent,

(c) contacting at least a portion of the raw material in consolidated form with the solution to swell the polyethylene and diffuse the biocompatible hydrophobic carrier and the beneficial agent into at least a portion of the raw material in consolidated form, and

(d) irradiating at least a portion of the raw material in consolidated form from (c) to crosslink at least a portion of the polyethylene.

The raw material in consolidated form, the solution comprising a biocompatible hydrophobic carrier and a beneficial agent, contacting conditions, irradiating conditions, and other treatments are as described above in paragraphs [0014]-[0024], except that the raw material in consolidated form is contacted with the solution comprising the beneficial agent and the biocompatible hydrophobic carrier first followed by irradiation of the thus contacted raw material in consolidated form.

The raw material in consolidated form produced in (c) or (d) above can be dried, e.g., under vacuum, to remove excess biocompatible hydrophobic carrier or beneficial agent and yield a raw material in consolidated form having a desired concentration of biocompatible hydrophobic carrier and the agent.

It is contemplated that the bearing material of the invention can have a number of uses. For example, the bearing material can be a prosthetic acetabular cup, an insert or liner of the acetabular cup, a trunnion bearing or a component thereof, a prosthetic tibial plateau, a patellar button, a prosthetic talar surface, a prosthetic radio-humeral joint, an ulno-humeral joint, a glenoro-humeral articulation, an intervertebral disk replacement, a facet joint replacement, a temporo-mandibular joint, or a finger joint. The bearing material may find use in the hip, knee, and extremity joints. The bearing material can be a liner for the acetabular component of a hip arthroplasty or the tibial bearing for a knee arthroplasty.

The orthopaedic bearing material or implant of the invention, can find use as a prosthesis for any suitable part of the body, e.g., such as a component of a joint in the body. For example, in a hip joint, the orthopaedic bearing material or implant can be a prosthetic acetabular cup, or the insert or liner of the cup, or a component of a trunnion bearing (e.g., between the modular head and the stem). In a knee joint, the orthopaedic bearing material or implant can be a prosthetic tibial plateau (femoro-tibial articulation), a patellar button (patello-femoral articulation), a trunnion or other bearing component, depending on the design of the artificial knee joint. For example, in a knee joint of the meniscal bearing type, both the upper and lower surfaces of the orthopaedic bearing material or implant, i.e., those surfaces that articulate against metallic or ceramic surfaces, may be surface-crosslinked. In an ankle joint, the orthopaedic bearing material or implant can be the prosthetic talar surface (tibio-talar articulation) or other bearing component. In an elbow joint, the orthopaedic bearing material or implant can be the prosthetic radio-humeral joint, the ulno-humeral joint, or other bearing component. In a shoulder joint, the orthopaedic bearing material or implant can be used in the glenoro-humeral articulation. In the spine, the orthopaedic bearing material or implant can be used in intervertebral disk replacement or facet joint replacement. The orthopaedic bearing material or implant can also be made into a temporo-mandibular joint (jaw) or a finger joint. The orthopaedic bearing material can find use as an implant in any part of a body, such as the hip, knee, and extremities.

The following examples further illustrate the invention but, of course, should not be construed as in any way limiting its scope.

### EXAMPLE 1

This example demonstrates that a crosslinked UHMWPE containing a biocompatible hydrophobic carrier releases the carrier under cyclic loading conditions by a load activated mechanism. UHMWPE (GUR 1050) is irradiated with gamma radiation (50 KGy or 5 Mrad) and melt annealed to quench free radicals. An AMTI Ortho-POD Wear Test Machine is used for the test. The test samples are ⅜ inch in diameter and 0.7 inch long irradiated UHMWPE pins that are soaked in squalene at 110° C. for 8 hours. The samples are sonic cleaned and dried at 50° C. The test is conducted such that the pins are dynamically loaded without articulation against a counterface to induce wear, so the results relate to the release of the biocompatible hydrophobic carrier only. A Paul loading curve, commonly used in hip stimulators, with a peak load of about 330 Newtons is applied to the pins. The cycling frequency is 1.66 Hz. A 90% bovine calf serum, diluted with EDTA and sodium azide dissolved in reverse osmosis water, is used as lubricant. The temperature of the lubricant is 37° C. During the dynamic loading test, the samples are soaked in serum without load for 2, 35, or 4 days between various test intervals.

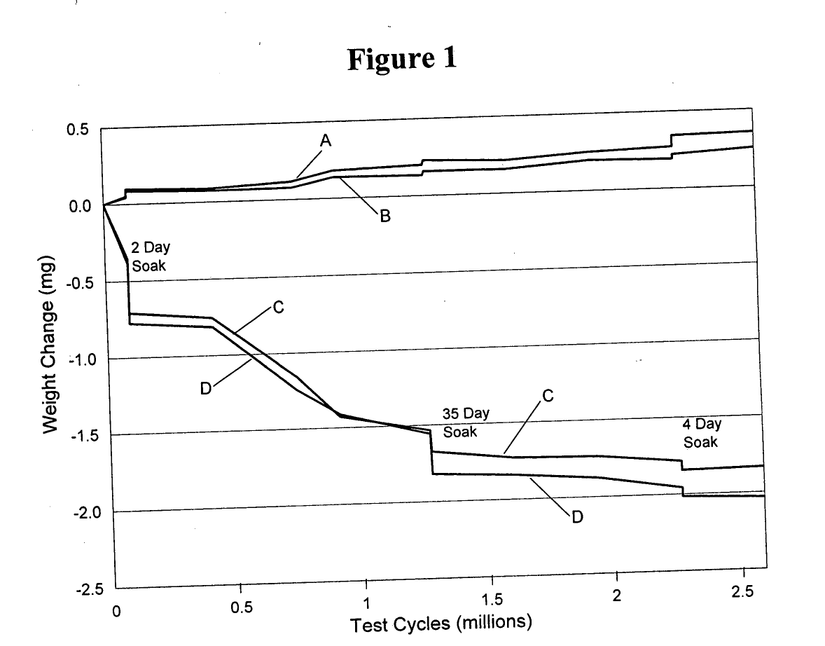

The weight change of the pins as a function of test cycles during the dynamic loading test is shown in FIG. 1. Control pins, without squalene (labeled A and B), show only a slight change in weight. The test pins (labeled C and D) show significant loss of weight with load, thereby indicating that the UHMWPE samples containing squalene releases the carrier under a load activated mechanism.

### EXAMPLE 2

This example demonstrates that a crosslinked UHMWPE sample containing a biocompatible hydrophobic carrier wears at substantially same rate as UHMWPE not containing the biocompatible hydrophobic carrier. A pin-on-disk (POD) wear test method is used to evaluate weight loss of the pin. An AMTI Ortho-POD Wear Test Machine is used for the test. UHMWPE (GUR 1050) is irradiated with gamma radiation (50 KGy or 5 Mrad) and melt annealed to quench free radicals. The test samples are ⅜ inch in diameter and 0.7 inch long irradiated UHMWPE pins that are soaked in squalene at 110° C. for 8 hours. The samples are sonic cleaned and dried at 50° C. A Paul loading curve with a peak load of about 330 Newtons is applied to the pins. The pins are moved against highly polished wrought CoCr disks in a 10×10 mm square pattern, synchronized with each loading cycle. The cycling frequency is 1.66 Hz. A 90% bovine calf serum, diluted with EDTA and sodium azide dissolved in reverse osmosis water, is used as lubricant. The temperature of the lubricant is 37° C. The wear test interval length is 330,000 cycles and the samples are weighed at the end of each interval.

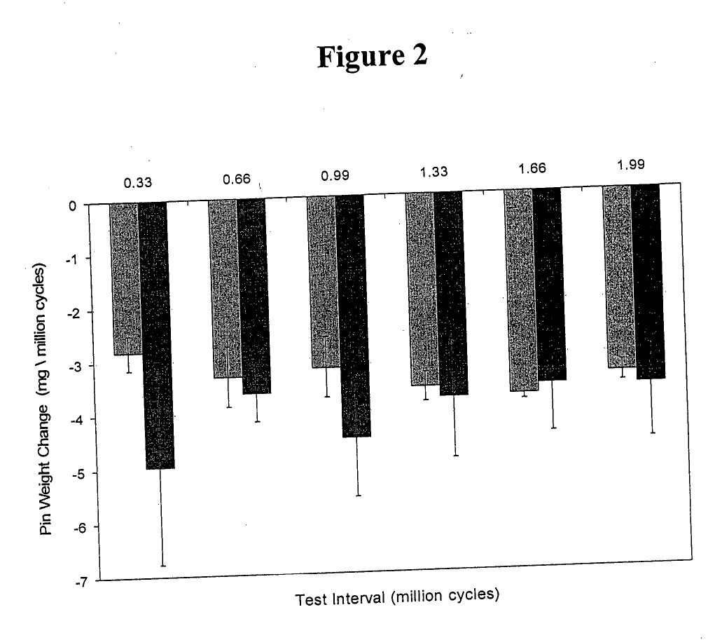

FIG. 2 shows the pin weight change in mg per million cycles during the wear test. For comparison, the weight change of control pins, without squalene, is also shown. In the early stages, the test pins lose more weight; however, as the number of cycles increases, the wear rate is substantially the same as the control pins. The rate of weight change stabilizes at 1.99 million cycles.

### EXAMPLE 3

This example demonstrates that an implant according to an embodiment of the invention releases the beneficial agent, beta-carotene, which is an antioxidant and also a model compound for a beneficial agent such as a drug, by a load-activated mechanism. UHMWPE (GUR 1050) is irradiated with gamma radiation (50 KGy or 5 Mrad) and melt annealed to quench free radicals. An AMTI Ortho-POD Wear Test Machine is used for the testing the UHMWPE cylindrical pin samples. The samples are ⅜ inch in diameter and 0.7 inch long, and are soaked in a 1.0 wt. % solution of beta-carotene in squalane at 90° C. for 24 hours. The samples are bright orange red in color, indicating the presence of beta-carotene.

A Paul loading curve with a peak load of about 330 Newtons is applied to the pins. The cycling frequency is 1.66 Hz. A 90% bovine calf serum, diluted with EDTA and sodium azide dissolved in reverse osmosis water, is used as lubricant. The temperature of the lubricant is 37° C. During the test, the samples become light orange in color, indicating that beta-carotene is being released, activated by the load.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A bearing material for a medical implant or medical implant part, the bearing material comprising: (a) a matrix of crosslinked polyethylene, (b) a biocompatible hydrophobic carrier, and (c) a beneficial agent soluble in the biocompatible hydrophobic carrier.

2. The bearing material of claim 1, wherein the biocompatible hydrophobic carrier is a lipid.

3. The bearing material of claim 1, wherein the biocompatible hydrophobic carrier is selected from the group consisting of squalane, squalene, benzyl benzoate, fatty acids, glycerides, polyisoprenoids, cholesterol, cholesterol esters, and any combination thereof.

4. The bearing material of claim 2, wherein the lipid is selected from the group consisting of squalane, squalene, and any combination thereof.

5. The bearing material of claim 1, wherein the beneficial agent is a drug, chemical, or biological.

6. The bearing material of claim 1, wherein the beneficial agent is selected from the group consisting of antibiotics, analgesics, anti-bone resorption drugs, bone growth factors, anti-cancer drugs, antioxidants, and any combination thereof.

7. The bearing material of claim 1, wherein the beneficial agent is beta-carotene.

8. The bearing material of claim 1, wherein the biocompatible hydrophobic carrier comprises about 0.01 wt. % to about 20 wt. % of the bearing material.

9. The bearing material of claim 8, wherein the biocompatible hydrophobic carrier comprises about 0.1 wt. % to about 10 wt. % of the bearing material.

10. The bearing material of claim 1, wherein the polyethylene is ultrahigh molecular weight polyethylene (UHMWPE).

11. A method for producing a bearing material for a medical implant or medical implant part, the method comprising:
(a) providing a raw material in consolidated form comprising polyethylene,
(b) irradiating at least a portion of the raw material in consolidated form to crosslink at least a portion of the polyethylene contained therein and form a matrix of crosslinked polyethylene,
(c) providing a solution comprising a biocompatible hydrophobic carrier and a beneficial agent soluble in the carrier, and
(d) contacting at least a portion of the matrix obtained in (b) with the solution to swell the polyethylene and diffuse the biocompatible hydrophobic carrier and the beneficial agent into the matrix.

12. The method of claim 11, wherein the polyethylene is ultrahigh molecular weight polyethylene having a weight average molecular weight of about 400,000 atomic mass units or more.

13. The method of claim 11, wherein the solution is maintained at a temperature of about 30° C. to about 150° C. during (d).

14. The method of claim 13, wherein the solution is maintained at a temperature of about 50° C. to about 120° C. during (d).

15. The method of claim 11, wherein the matrix is contacted with the solution for about 2 hours or more in (d).

16. The method of claim 11, wherein the biocompatible hydrophobic carrier is a lipid.

17. The method of claim 11, wherein the biocompatible hydrophobic carrier is selected from the group consisting of squalane, squalene, benzyl benzoate, fatty acids, glycerides, polyisoprenoids, cholesterol, cholesterol esters, and any combination thereof.

18. The method of claim 11, wherein the beneficial agent is a drug, chemical, or biological.

19. The method of claim 11, further comprising drying the matrix obtained in (d) to remove excess biocompatible hydrophobic carrier or beneficial agent.

20. A method for producing a bearing material for a medical implant or medical implant part, the method comprising:
(a) providing a raw material in consolidated form comprising polyethylene,
(b) providing a solution comprising a biocompatible hydrophobic carrier and a beneficial agent,
(c) contacting at least a portion of the raw material in consolidated form with the solution to swell the polyethylene and diffuse the biocompatible hydrophobic carrier and the beneficial agent into at least a portion of the raw material in consolidated form, and
(d) irradiating at least the portion of the raw material in consolidated form from (c) to crosslink at least a portion of the polyethylene contained therein and form a matrix of crosslinked polyethylene.
